(19) United States
(12) Patent Application Publication
(10) Pub. No.: US 2022/0142570 A1
(43) Pub. Date: May 12, 2022

(54) SYSTEM AND METHOD FOR A WEARABLE DEVICE TO MEASURE AND MONITOR HUMAN BODY VITALS

(71) Applicant: INBIOZ TECHNOLOGY PRIVATE LIMITED, KOLKATA (IN)

(72) Inventors: SOUVIK MAZUMDER, BANGALORE (IN); MOHAMMAD ZEESHAN, KAPTANGANJ (IN); ABHISHEK B M FERNANDES, BANGALORE (IN)

(73) Assignee: INBIOZ TECHNOLOGY PRIVATE LIMITED

(21) Appl. No.: 17/512,695

(22) Filed: Oct. 28, 2021

(30) Foreign Application Priority Data
Nov. 10, 2020 (IN) 202031049066

(51) Int. Cl. A61B 5/00; A61B 5/0531; A61B 5/0537; A61B 5/20

(52) U.S. Cl. CPC A61B 5/681; A61B 5/0531; A61B 5/0537; A61B 5/742; A61B 5/4519; A61B 5/201; A61B 5/0002; A61B 5/7257

(57) ABSTRACT

A system for a wearable device to measure and monitor human body vitals is provided. The wearable device includes a main body and a strap coupled to the main body. The main body includes a first electrode and a second electrode configured to contact the wrist of the user and a third electrode coupled at a top side of the main body. The main body includes a mechanical switch connected at a first position to supply voltage between first electrode and the second electrode to measure a skin impedance using an internal measurement unit. The mechanical switch is connected at a second position to supply voltage between the first electrode and the third electrode to measure full body impedance when a user touches the third electrode with another hand/leg. The processing subsystem calculates multiple human body vitals based on the calculated skin impedance and full body impedance.

### CROSS-REFERENCE TO RELATED APPLICATION

This application claims priority from a complete patent application filed in India having Patent Application No. 202031049066, filed on Nov. 10, 2020 and titled “SYSTEM AND METHOD FOR A WEARABLE DEVICE TO MEASURE AND MONITOR HUMAN BODY VITALS”

### FIELD OF INVENTION

Embodiments of the present disclosure relate to wearable devices and more particularly to a system and a method for a wearable device to measure and monitor human body vitals.

### BACKGROUND

The development of medical technologies and extension of the average lifespan, there has been an increasing interest in health care. There is a huge jump in awareness and consumption of preventive health care across all over the world in recent times. We are also seeing a growing trend in fitness wearable among a section of population in India and abroad. In particular, athletes and patients, among a number of other consumers, are key individuals who require accurate and up-to-date (i.e. real-time) body vital data.

Furthermore, some of the body vitals measuring device, which is a sort of health care device, measures body compositions using a bioelectrical impedance analysis, In this BIA method, an alternating low amplitude current is being applied to the human body and by measuring the electrical impedance of human body, different body compositions can be calculated by using some empirical formulae. However, such BIA devices utilize a typical sensor arrangement which can be bulky and uncomfortable for the typical wearer. In short, these market available BIA devices are neither wearable not affordable.

Hence, there is a need for an improved system and method in the form of a wearable device to measure and monitor human body vitals to address the aforementioned issue(s).

### BRIEF DESCRIPTION

In accordance with an embodiment of the present disclosure, a system for wearable device to measure different body compositions and monitor body hydration is provided. The system includes the wearable device. The wearable device includes a main body and a strap coupled to the main body. The strap is configured to be coupled to the wrist of a user. The main body includes a first electrode and a second electrode coupled at a bottom side of the main body. The first electrode and the second electrode are configured to touch the skin of the user around his/her wrist. The main body also includes a third electrode coupled at a top side of the main body. The main body further includes a mechanical switch configured to enable a voltage supply between the first electrode, the second electrode and the third electrode in a predefined combination. The combination includes a first position of the mechanical switch when the voltage is applied between the first electrode and the second electrode. The combination also includes a second position of the mechanical switch when the voltage is applied between the first electrode and the third electrode. There is an internal measurement unit inside the main body which is configured to measure the external impedance between the bottom two electrodes when the mechanical switch is in first position. Similarly, the internal measurement unit is configured to measure the external impedance connected between first electrode and the third electrode when the mechanical switch is in second position. Now the internal measurement unit can measure the skin impedance of the user when mechanical switch is in first position and the user wears the device around his wrist and the internal measurement unit can measure the full body impedance from wrist to ankle of the user when the mechanical switch is in second position and user touches the third electrode to his ankle.

The system includes a processing subsystem hosted on a server and in communication with the internal measuring unit. The processing subsystem is configured to determine extracellular fluid and intracellular fluid at a plurality of frequencies by converting the skin impedance and the full body impedance measured by the internal measuring unit. The processing subsystem is also configured to calculate the plurality of human body vitals based on determined extracellular fluid and intracellular fluid using one or more empirical formulas, wherein the plurality of human body vitals comprises at least one of body fat, protein and minerals, muscle mass, hydration status or a combination thereof. The processing subsystem is further configured to generate a plurality of personalized recommendations associated with the fitness of the user based on a plurality of calculated human body vitals and fitness requirement of the user.

In accordance with another embodiment of the present disclosure, a method to operate for a wearable device to measure and monitor human body vitals is provided. The method includes enabling, by a mechanical switch, a voltage supply between a first electrode, a second electrode and a third electrode in a predefined combination, where the first electrode and the second electrode are coupled at a bottom side of the main body to contact wrist of a user and a third electrode coupled at a top side of the main body. The method also includes changing a position of the mechanical switch to a first position to apply voltage between the first electrode and the second electrode. The method further includes measuring, by an internal measurement unit, a skin impedance including the skin/electrode interface impedance [for two electrodes] of the user when the mechanical switch is in the first position. The method further includes changing a position of the mechanical switch to a second position to apply voltage between the first electrode and the third electrode. The method further includes measuring, by the internal measurement unit, a full body impedance of the user along with the skin/electrode interface impedance [for two electrodes] when the user touches his ankle with the third electrode and the mechanical switch is in second position.

The method further includes determining, by a processing subsystem, extracellular fluid and intracellular fluid at a plurality of frequencies by converting the skin impedance, and the full body impedance measured by the internal measuring unit. The method further includes calculating, by the processing subsystem, the plurality of human body vitals based on determined extracellular fluid and intracellular fluid using one or more empirical formulas, where the plurality of human body vitals comprises at least one of body fat, protein and minerals, muscle mass, hydration status or a combination thereof. The method further includes generating, by the processing subsystem, a plurality of personalized recommendations associated with the fitness of the user based on a plurality of calculated human body vitals and fitness requirement of the user.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a system for a wearable device to measure and monitor human body vitals in accordance with an embodiment of the present disclosure;

FIGS. 2(a) and 2(d) are schematic representation of internal measurement unit of FIG. 1 and FIGS. 2(c) and 2(f) are schematic representation of position of mechanical switch of FIG. 1 in accordance with an embodiment of the present disclosure and FIGS. 2(b) and 2(e) are representing of how different electrodes are touching different parts of human body while measuring skin and full body impedance;

FIG. 3 is a schematic representation of perspective views of wearable device of FIG. 1 in accordance with an embodiment of the present disclosure;

FIG. 4 is a schematic representation of an exemplary embodiment of the system of FIG. 1 in accordance with an embodiment of the present disclosure;

FIGS. 5(a) and 5(b) is a flow chart representing the steps involved in a method for wearable device to calculate human body vitals in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a system and method for wearable device to measure and monitor human body vitals is provided. The system includes the wearable device. The wearable device includes a main body and a strap coupled to the main body. The strap is configured to be coupled to a wrist of a user. The main body includes a first electrode and a second electrode coupled at a bottom side of the main body. The first electrode and the second electrode are configured to touch the wrist of the user. The main body also includes a third electrode coupled at a top side of the main body. The main body further includes a mechanical switch configured to enable a voltage supply between either first and third electrode or first and second electrode. The combination includes a first position of the mechanical switch when the voltage is applied between the first electrode and the second electrode. The combination also includes a second position of the mechanical switch when the voltage is applied between the first electrode and the third electrode. There is an internal measurement unit inside the main body which is configured to measure the external impedance between the bottom two electrodes when the mechanical switch is in first position. Similarly, the internal measurement unit is configured to measure the external impedance connected between first electrode and the third electrode when the mechanical switch is in second position. Now the internal measurement unit can measure the skin impedance of the user including skin/electrode interface impedance [for two electrodes] when mechanical switch is in first position and the user wears the device around his wrist and the internal measurement unit can measure the full body impedance from wrist to ankle of the user along with the skin/electrode interface impedance [for two electrodes] when the mechanical switch is in second position and user touches the third electrode to his ankle. The system includes a processing subsystem hosted on a server and in communication with the internal measuring unit. The processing subsystem is configured to determine extracellular fluid and intracellular fluid at a plurality of frequencies by converting the skin impedance and the full body impedance measured by the internal measuring unit. The processing subsystem is also configured to calculate the plurality of human body vitals based on determined extracellular fluid and intracellular fluid using one or more empirical formulas, wherein the plurality of human body vitals comprises at least one of body fat, protein and minerals, muscle mass, hydration status or a combination thereof. The processing subsystem is further configured to generate a plurality of personalized recommendations associated with the fitness of the user based on a plurality of calculated human body vitals and fitness requirement of the user.

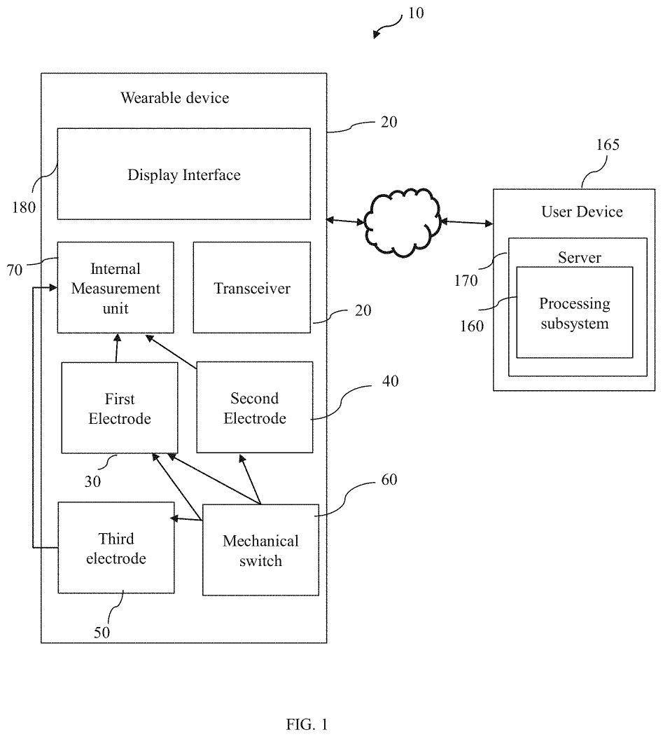

FIG. 1 is a block diagram representation of a system 10 for a wearable device 20 to measure and monitor human body vitals in accordance with an embodiment of the present disclosure. The system 10 includes a wearable device 20 which further includes a first electrode 30 and a second electrode 40 coupled at a bottom side of a main body of the wearable device. The first electrode and the second electrode are configured to contact the skin around the wrist of the user. The wearable device also includes a third electrode 50 coupled at a top side of the main body. As used herein, the electrode is an electrical conductor used to make an electrical contact with any external impedance. The electrodes are used to excite the external impedance with an alternating current of known amplitude. The electrodes are made of some kind of metal or metallic alloy. The wearable device further includes a mechanical switch 60 which is configured to enable a voltage supply between the first electrode, the second electrode and the third electrode in a predefined combination. The mechanical switch attains a first position when the voltage is applied between the first electrode and the second electrode. The mechanical switch attains a second position when the voltage is applied between the first electrode and the third electrode. As used herein, the mechanical switch may be a linear switch, a tactile switch, clicky switch or the like.

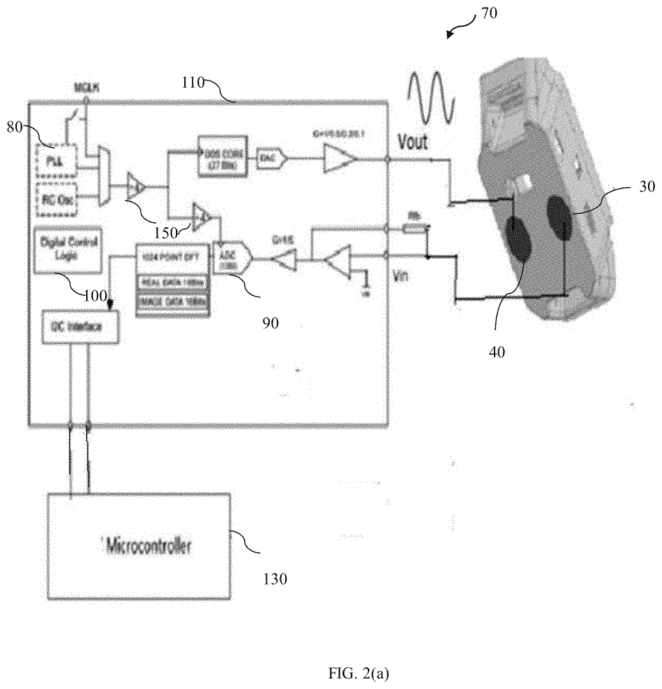

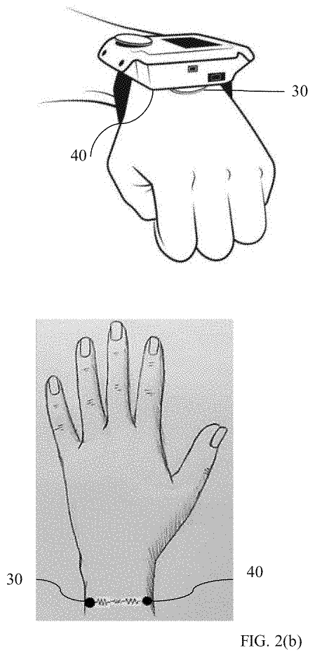

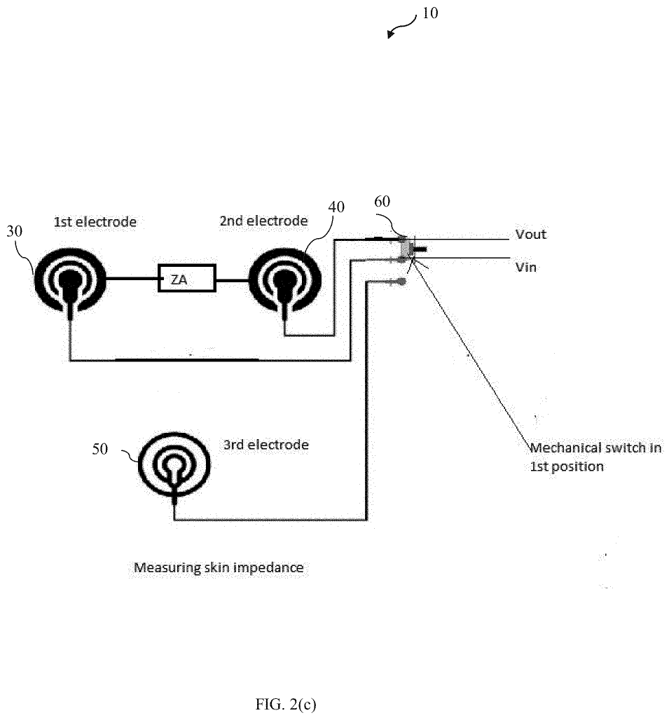

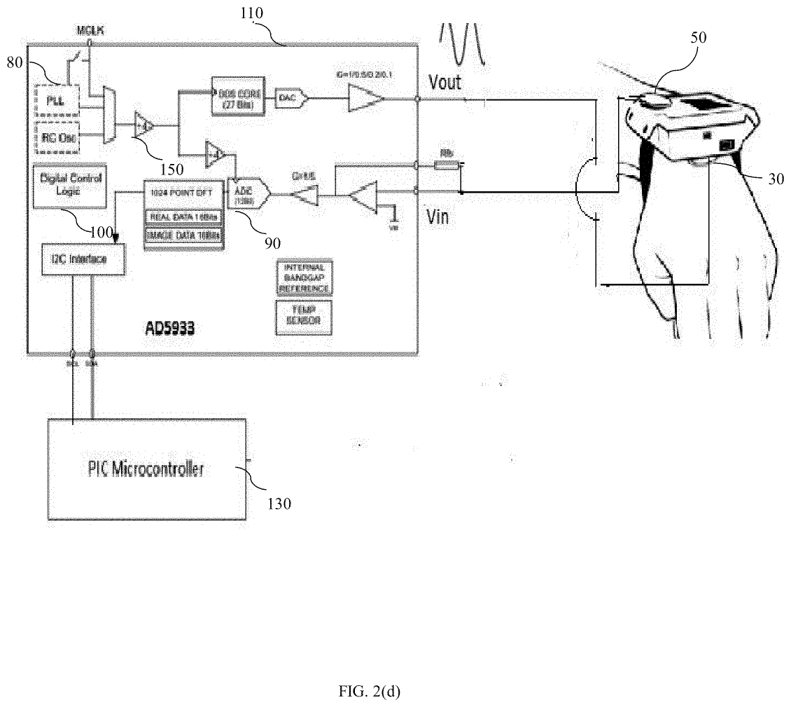

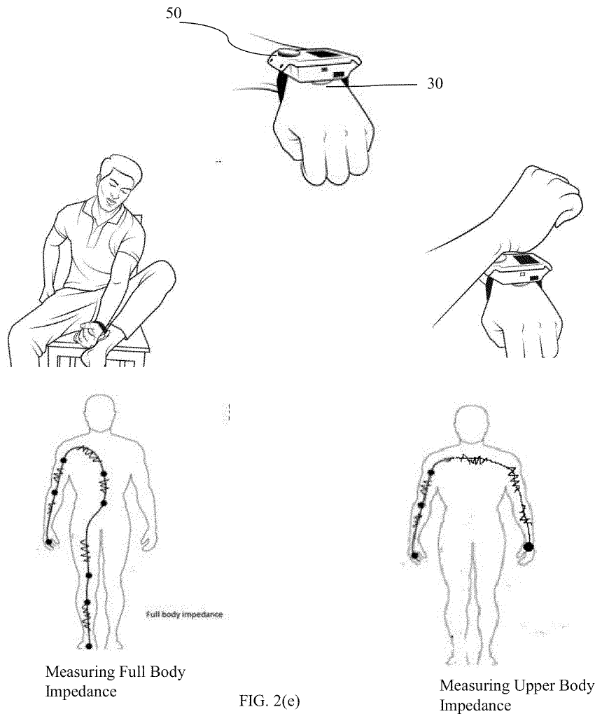

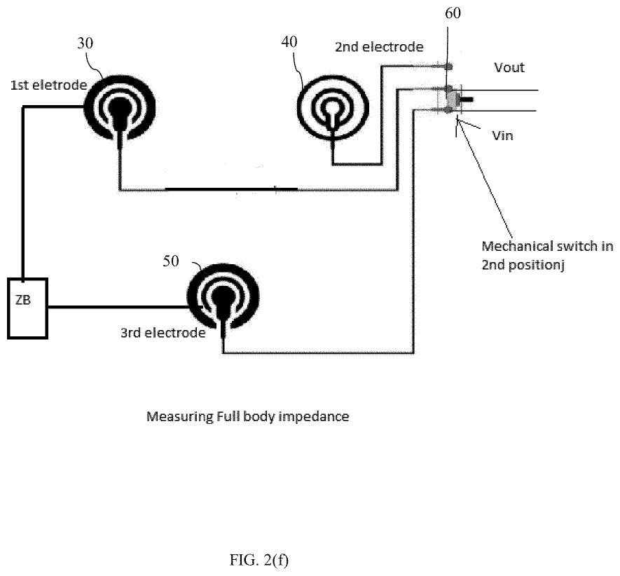

Furthermore, the wearable device further includes an internal measurement unit 70 electrically coupled to the first electrode, the second electrode and the third electrode. The internal measurement unit is configured to measure a skin impedance of a body of the user when the mechanical switch is in the first position. The internal measurement unit is further configured to measure a full body impedance of the user when the user touches an ankle with the third electrode and the mechanical switch is in second position. In one embodiment, the internal measurement unit may be configured to measure at least one of pulse rate, heartbeat, body temperature or a combination thereof. One embodiment of the internal measurement unit is described in FIG. 2 in detail.

FIG. 2 is a schematic representation of internal measurement unit 70 of FIG. 1 in accordance with an embodiment of the present disclosure. When the mechanical switch is in the first position and voltage is applied between first electrode and the second electrode, the internal measurement unit may measure skin impedance including the skin and an electrode interface impedance [for two electrodes] as shown in FIGS. 2(a), 2(b) and 2(c). When the mechanical switch is in second position and voltage is applied between the first electrode and the third electrode, the internal measurement unit may measure full body impedance along with the skin/electrode interface impedance [for two electrodes] as shown in FIGS. 2(d), 2(e) and 2(f). The internal measurement unit includes a frequency generator 80 which is configured to enable an external complex impedance to be excited with a known frequency. The internal measurement unit also includes an analog to digital converter (ADC) 90 which is configured to sample a response signal from the excited external complex impedance. The internal measurement unit further includes a digital signal processing engine 100 which is configured to process sampled response signal using a Fast Fourier Transform (FFT) model. The FFT model returns a real and imaginary data and thereby enabling the external impedance to be calculated. This external impedance may be skin or full body impedance of the user depending on the position of the mechanical switch and where the user touches the third electrode.

In detail, the internal measurement unit includes a printed circuit board (PCB) 110, a microcontroller 130, a transceiver (140, FIG. 1), a power regulator IC, an OPAMP IC 150, a crystal oscillator and few resistors and capacitors. In a specific embodiment, the transceiver of the wearable device may be a Bluetooth enabled device with an easy-to-use ASCII-style command interface. The transceiver is configured to send the skin impedance and the full body impedance measured by the internal measuring unit to a processing subsystem. Similarly, the transceiver is also configured to receive the plurality of human body vitals calculated by the processing subsystem. In one embodiment, the transceiver communicates with the user device in a wired or wireless manner. For example, the transceiver may communicate with the user device using at least one of the communication protocols including, but not limited to, Bluetooth® communication, Bluetooth@ low energy (BLE) communication, near field communication (NFC), wireless local area network (WLAN) communication, wireless fidelity (WiFi) communication, ZigBee communication, infrared data association (IrDA) communication, Wi-Fi direct (WFD) communication, ultra-wideband (UWB) communication, Ant+ communication, and the like. In some embodiments, the user device may be a mobile phone, a laptop, a tablet, a computer or the like.

Referring back to FIG. 1, the system 10 includes a processing subsystem 160 hosted on a server 170 and in communication with the internal measuring unit. In one embodiment, the server may include a local server. In another embodiment, the server may include a cloud-based server. The server may be operated on the user device 165 associated with the user. The processing subsystem 160 may be a hardware for controlling the overall function and operation of the wearable device. The processing subsystem executes the program stored in the memory to analyze the human body vitals using the skin and body impedance measured by the internal measurement unit. The memory may also store additional data such as the height, weight, and gender of the user, and the like.

The processing subsystem is configured to determine extracellular fluid and intracellular fluid at a plurality of frequencies by converting the skin impedance and the full body impedance measured by the internal measuring unit. As used herein, the extracellular fluids (ECF) denote all body fluid outside the cells of any multicellular organism. The extracellular fluid makes up about one-third of body fluid, the remaining two-thirds is intracellular fluid within cells. The main component of the extracellular fluid is the interstitial fluid that surrounds cells. Similarly, the intracellular fluid is the fluid that exists within the cells of multi-celled organisms. The intracellular fluid is therefore stored within the intracellular compartments of the body. The processing subsystem is also configured to calculate the plurality of human body vitals based on determined extracellular fluid and intracellular fluid using one or more empirical formulas. The plurality of human body vitals comprises at least one of body fat, protein and minerals, muscle mass, hydration status or a combination thereof. Consider a non-limiting exemplary calculation of the various human body vitals using the measured skin impedance and full body impedance.

Z1=Skin Impedance, Z2=Full Body Impedance, where Z2 and Z1 are measured in terms of real and imaginary values at 50 Kilohertz frequency.
Z1=2×Z1 [electrode/human skin interface impedance]+Z2 [skin impedance between two bottom electrodes 1 inch apart]
ZB=2×Z1 [electrode human skin interface impedance]+Z3 [body impedance from wrist to ankle]+Z4 impedance of the connector cable [if any]

Then, Effective Body Impedance [Z]=ZB−ZA equation (1)

[where Z2 and Z4 are considered to be almost negligible]

Fat Free Mass FFM is calculated as:

For Male,

FFM=−10.68+0.65 height2/Z+0.26 weight+0.02 Z equation (2)

For Female,

FFM=−9.53+0.69 height2/Z+0.17 weight+0.02 Z equation (3)

where FFM is in Kg, height²/resistance is in cm²/ohms, and resistance is in ohms

Based on the calculation of fat free mass (FFM), fat mass, body fat %, muscle mass and total body water of user may also be calculated. In a specific embodiment, the processing subsystem may be configured to determine extracellular fluid excess by measuring body impedance at the plurality of frequencies and thereby provide an independent predictor of overall renal function, chronic kidney diseases and cardiovascular morbidity in patients undergoing dialysis.

The processing subsystem is further configured to generate a plurality of personalized recommendations associated with the fitness of the user based on a plurality of calculated human body vitals and fitness requirement of the user. Based on the body fat, protein and minerals, muscle mass, hydration status or like, the processing subsystem generates multiple recommendations which is personalized for each user.

In one embodiment, the wearable device 20 may include a display interface 180 configured to display the human body vitals calculated by the processing subsystem to the user. The main body of the wearable device may include a button, a switch, the display interface, or the like via which the user may operate the wearable device. The display interface may include a display panel such as a liquid crystal display (LCD) panel, an organic light-emitting diode (OLED) panel, or the like. The display panel displays information about the analyzed body vitals result in the form of an image or a text. In a specific embodiment, the main body of the wearable device may include a separate battery (not shown in FIG. 1) which supplies power to PCB along with a charger circuit PCB. In some embodiments, the main body may include a silicon plate with a layer of silver chloride (AgCl) between the first electrode, the second electrode, third electrode and skin for better electrical conductivity.

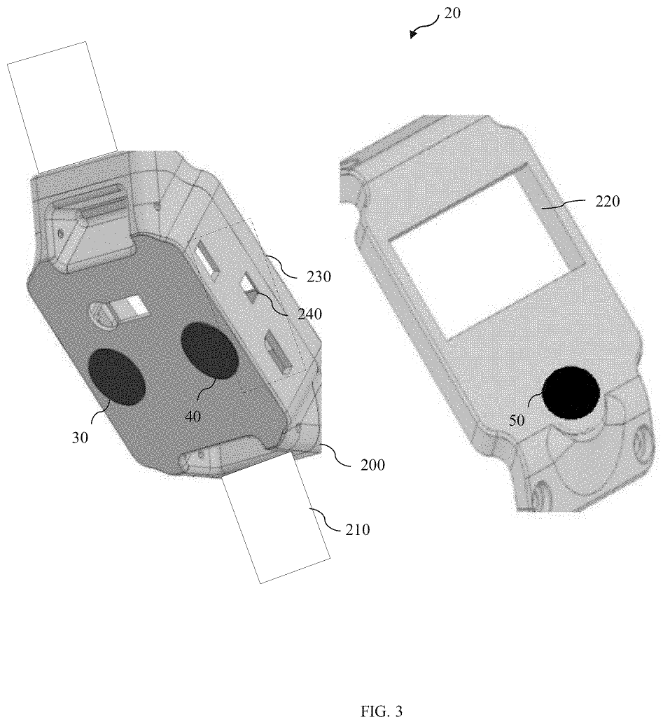

FIG. 3 is a schematic representation of perspective views of wearable device 20 of FIG. 1 in accordance with an embodiment of the present disclosure. The system 10 includes a wearable device 20 having a main body 200 and a strap 210 which is coupled to the main body. The strap is configured to be coupled to a wrist of a user. More specifically, the strap is provided as two part straps at both sides of the main body, in a way that the strap is connected with the main body and is wearable on the wrist of the user. In one embodiment, the main body includes two circular holes at the bottom surface of the main body. The two circular holes are adapted to receive a first electrode 30 and a second electrode 40. The two holes are provided in such a way that the first electrode and the second electrode touch the skin of the user. In such an embodiment, the main body includes one circular hole on the top surface of the main body. The one circular hole is adapted to receive a third electrode 50 which a user may touch with another hand/ankle when required. In some embodiments, the main body also includes a rectangular hollow space 220 on the top surface of the main body. The rectangular hollow space is adapted to receive a display interface. In a specific embodiment, the main body include a plurality of holes 230 on the side of the main body, where one hole 240 from the plurality of holes is adapted to receive a mechanical switch.

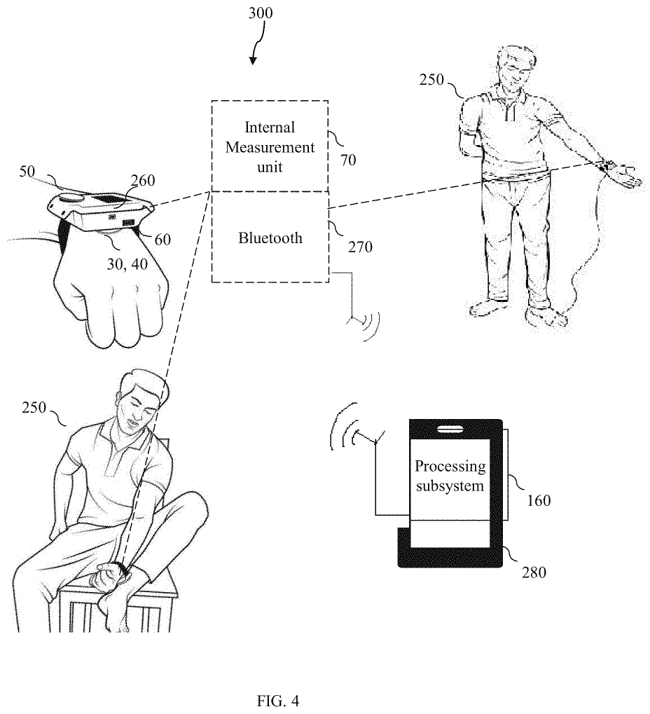

FIG. 4 is a schematic representation of an exemplary embodiment of the system 10 of FIG. 1 in accordance with an embodiment of the present disclosure. Considering a non-limiting example where a user X 250 wears a wearable device on the left wrist. The user initializes the wearable device using a push button 260. Once the wearable device is in operating condition, the user put the mechanical switch 60 (placed on the main body of the wearable device) in down position so that voltage is being applied between bottom two electrodes 30, 40 (first electrode and the second electrode) to take the skin impedance measurement via the internal impedance measurement unit 70 of the wearable device between two bottom electrodes.

Furthermore, the user put the mechanical switch in upward position so that voltage is being applied between one bottom electrode (first electrode) 30 and top electrode (third electrode) 50. Now, when the user touches his other right hand (more specifically, wrist) or leg (more specifically, ankle) with top electrode or use some cable to connect the top electrode to his ankle to take impedance measurement between top and one bottom electrode and hence upper or full body impedance is calculated via the internal measurement unit of the wearable device.

Subsequently, the Bluetooth based transceiver 270 placed in main body of the wearable device sends all these impedance data (skin impedance, upper body impedance or full body impedance) to user's mobile phone 280. Further, the processing subsystem 160 running on the mobile phone of the user calculates effective body impedance from skin and full body impedance. Here, the processing subsystem may be used to run the application programs via APIs. Once the processing subsystem generates effective body impedance, the processing subsystem calculates the total body fluid and fat free mass of user using some standard equations and then calculates body fat mass, muscle mass, fat % or the like.

The processing subsystem also calculates extracellular fluid excess by measuring body impedance at low and high frequency and thus give an independent predictor of overall renal function and of cardiovascular morbidity in patients undergoing dialysis. Also, the processing subsystem measures any imbalance in intra and extra cellular water in human body and thus give an independent indictor of overall renal function and stages of CKD (Chronic Kidney diseases). The wearable device uses solid silicon plate (which may be detachable with a thin layer of AgCl between metal electrodes and skin for better electrical conductivity.

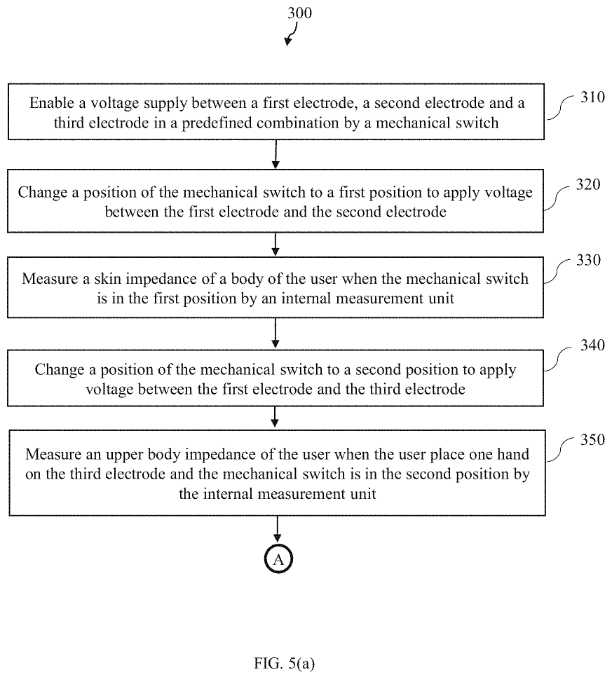

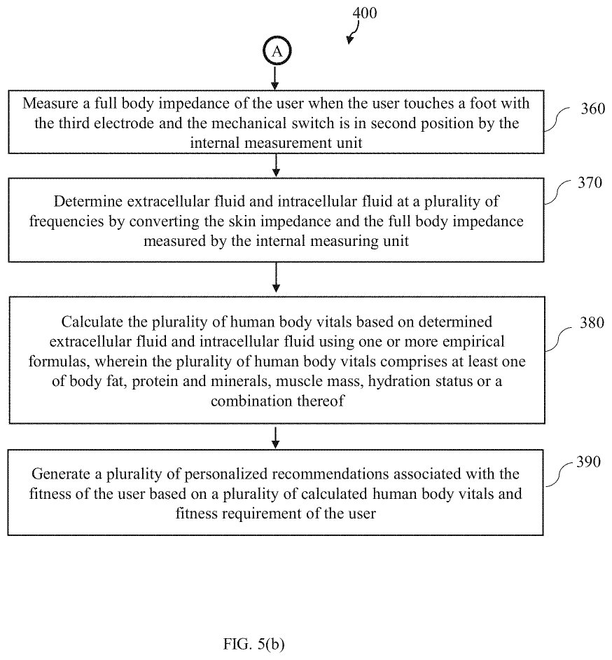

FIGS. 5(a) and 5(b) illustrates a flow chart representing steps involved in a method 300 for wearable device to calculate human body vitals in accordance with an embodiment of the present disclosure. The method includes enabling, by a mechanical switch, a voltage supply between a first electrode, a second electrode and a third electrode in a predefined combination in step 310, where the first electrode and the second electrode are coupled at a bottom side of the main body to contact wrist of a user and a third electrode coupled at a top side of the main body.

The method 300 also includes changing a position of the mechanical switch to a first position to apply voltage between the first electrode and the second electrode in step 320. The method 300 further includes measuring, by an internal measurement unit, a skin impedance of a body of the user when the mechanical switch is in the first position in step 330. In one embodiment, measuring, by an internal measurement unit, a skin impedance of a body of the user includes enabling an external complex impedance to be excited with a known frequency using a frequency generator. In such an embodiment, measuring a skin impedance of a body of the user may include sampling a response signal from the excited external complex impedance using an analog to digital converter. In such another embodiment, measuring a skin impedance of a body of the user may include processing sampled response signal using a Fast Fourier Transform (FFT) model using a digital signal processing engine, wherein the FFT model returns a real and imaginary data and thereby enabling the skin impedance to be calculated.

The method 300 further includes changing a position of the mechanical switch to a second position to apply voltage between the first electrode and the third electrode in step 340. The method 300 further includes measuring, by the internal measurement unit, an upper body impedance of the user when the user place one hand on the third electrode and the mechanical switch is in the second position in step 350. The method 300 further includes measuring, by the internal measurement unit, a full body impedance of the user when the user touches an ankle with the third electrode and the mechanical switch is in second position in step 360. In a specific embodiment, correct the body impedance based on an electrode interface impedance and calculate the human body vitals based on a corrected body impedance. In one embodiment, the method 300 includes sending the skin impedance, the upper body impedance and the full body impedance measured by the internal measuring unit to the processing subsystem via a transceiver.

The method 300 further includes determining, by a processing subsystem, extracellular fluid and intracellular fluid at a plurality of frequencies by converting the skin impedance and the full body impedance measured by the internal measuring unit in step 370. In one embodiment, the method 300 may include receiving the plurality of human body vitals calculated by the processing subsystem via a transceiver. In a specific embodiment, determining the extracellular fluid excess by measuring body impedance at the plurality of frequencies and thereby provide an independent predictor of overall renal function, chronic kidney diseases and cardiovascular morbidity in patients undergoing dialysis.

The method 300 further includes calculating, by the processing subsystem, the plurality of human body vitals based on determined extracellular fluid and intracellular fluid using one or more empirical formulas, where the plurality of human body vitals comprises at least one of body fat, protein and minerals, muscle mass, hydration status or a combination thereof in step 380. The method 300 further includes generating, by the processing subsystem, a plurality of personalized recommendations associated with the fitness of the user based on a plurality of calculated human body vitals and fitness requirement of the user in step 390. In one embodiment, the method may include displaying the plurality of human body vitals calculated by the processing subsystem via a display interface.

Various embodiments of the system and method for a wearable device to measure and monitor human body vitals as described above enables measuring body compositions such as fat %, muscle mass, body water or the like and providing some health marker for preventive diagnosis by using a wearable device. The system helps in detecting acute dehydration state and any imbalance in intra and extra cellular water by using the wearable device. The design and arrangement of the electrodes enable the user convenience for measuring body vitals. The system empowers people with the information they need to better manage their health and the health of their loved ones.

The system may address the data storage requirements for health and wellness management, chronic disease management or patient recovery, medication management, and fitness and workout tracking. For example, a sport or fitness enthusiast may desire to monitor, collect, and/or analyze various aspects of the fitness routine (such as their heart rate, workout intensity, workout duration, and so forth) to determine how to improve and adjust their fitness routine to increase its efficacy.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown: nor do all of the acts need to be necessarily performed. Also, those acts that are not dependent on other acts may be performed in parallel with the other acts. The scope of embodiments is by no means limited by these specific examples.

We claim:

1. A system for a wearable device to measure a plurality of human body vitals comprising:
the wearable device, wherein the wearable device comprises:
a main body; and
a strap coupled to the main body, wherein the strap is configured to be coupled to a wrist of a user;
characterized by
a first electrode and a second electrode coupled at a bottom side of the main body, wherein the first electrode and the second electrode are configured to contact the wrist of the user;
a third electrode coupled at a top side of the main body;
a mechanical switch configured to enable a voltage supply between the first electrode, the second electrode and the third electrode in a predefined combination, wherein the combination comprises:
a first position of the mechanical switch when the voltage is applied between the first electrode and the second electrode; and
a second position of the mechanical switch when the voltage is applied between the first electrode and the third electrode;
an internal measurement unit electrically coupled to the first electrode, the second electrode and the third electrode, the internal measurement unit is configured to:
measure a skin impedance of a body of the user when the mechanical switch is in the first position;
measure an upper body impedance of the user when the user place one hand on the third electrode and the mechanical switch is in the second position; and
measure a full body impedance of the user when the user touches an ankle with the third electrode and the mechanical switch is in second position; and
a processing subsystem hosted on a server and in communication with the internal measuring unit, the processing subsystem is configured to:
determine extracellular fluid and intracellular fluid at a plurality of frequencies by converting the skin impedance and the full body impedance measured by the internal measuring unit;
calculate the plurality of human body vitals based on determined extracellular fluid and intracellular fluid using one or more empirical formulas,
wherein the plurality of human body vitals comprises at least one of body fat, protein and minerals, muscle mass, hydration status or a combination thereof; and
generate a plurality of personalized recommendations associated with the fitness of the user based on a plurality of calculated human body vitals and fitness requirement of the user.

2. The system as claimed in claim 1, wherein the internal measurement unit is configured to measure at least one of pulse rate, heartbeat, body temperature or a combination thereof.

3. The system as claimed in claim 1, wherein the internal measurement unit comprises:
a frequency generator configured to enable an external complex impedance to be excited with a known frequency;
an analog to digital converter (ADC) is configured to sample a response signal from the excited external complex impedance; and
a digital signal processing engine is configured to process sampled response signal using a Fast Fourier Transform (FFT) model, wherein the FFT model returns a real and imaginary data and thereby enabling the external impedance to be calculated.

4. The system as claimed in claim 1, wherein the processing subsystem is configured to determine the extracellular fluid excess by measuring body impedance at the plurality of frequencies and thereby provide an independent predictor of overall renal function, chronic kidney diseases and cardiovascular morbidity in patients undergoing dialysis.

5. The system as claimed in claim 1, wherein the processing subsystem is configured to correct the body impedance based on an electrode interface impedance and calculate the human body vitals based on a corrected body impedance.

6. The system as claimed in claim 1, wherein the main body comprises silicon or silicon alloy three electrodes to provide better electrical conductivity with the human skin.

7. The system as claimed in claim 1, wherein the main body comprises a transceiver configured to:
send the skin impedance, the upper body impedance and the full body impedance measured by the internal measuring unit to the processing subsystem; and
receive the plurality of human body vitals calculated by the processing subsystem.

8. The system as claimed in claim 1, wherein the main body comprises a display interface configured to display the plurality of human body vitals calculated by the processing subsystem.

9. A method comprising:
enabling, by a mechanical switch, a voltage supply between a first electrode, a second electrode and a third electrode in a predefined combination,
wherein the first electrode and the second electrode are coupled at a bottom side of the main body to contact wrist of a user and a third electrode coupled at a top side of the main body;
changing a position of the mechanical switch to a first position to apply voltage between the first electrode and the second electrode;
measuring, by an internal measurement unit, a skin impedance of a body of the user when the mechanical switch is in the first position;
changing a position of the mechanical switch to a second position to apply voltage between the first electrode and the third electrode;
measuring, by the internal measurement unit, an upper body impedance of the user when the user place one hand on the third electrode and the mechanical switch is in the second position;
measuring, by the internal measurement unit, a full body impedance of the user when the user touches an ankle with the third electrode and the mechanical switch is in second position;
determining, by a processing subsystem, extracellular fluid and intracellular fluid at a plurality of frequencies by converting the skin impedance and the full body impedance measured by the internal measuring unit;
calculating, by the processing subsystem, the plurality of human body vitals based on determined extracellular fluid and intracellular fluid using one or more empirical formulas, wherein the plurality of human body vitals comprises at least one of body fat, protein and minerals, muscle mass, hydration status or a combination thereof; and
generating, by the processing subsystem, a plurality of personalized recommendations associated with the fitness of the user based on a plurality of calculated human body vitals and fitness requirement of the user.

10. The method as claimed in claim 9, wherein measuring, by an internal measurement unit, a skin impedance of a body of the user comprises:
enabling an external complex impedance to be excited with a known frequency using a frequency generator;
sampling a response signal from the excited external complex impedance using an analog to digital converter; and
processing sampled response signal using a Fast Fourier Transform (FFT) model using a digital signal processing engine, wherein the FFT model returns a real and imaginary data and thereby enabling the external impedance which is skin or full body impedance of the user to be calculated.
